(19) United States
(12) Patent Application Publication
(10) Pub. No.: US 2024/0058625 A1
(43) Pub. Date: Feb. 22, 2024

(54) FLUID CHANNEL STRUCTURE OF NON-INVASIVE PROSTHETIC DEVICE

(71) Applicant: University of Southern California, Los Angeles, CA (US)

(72) Inventors: Qifa Zhou, Arcadia, CA (US); Mark S. Humayun, Los Angeles, CA (US); Gengxi Lu, Los Angeles, CA (US)

(21) Appl. No.: 18/210,978

(22) Filed: Jun. 16, 2023

Related U.S. Application Data
(60) Provisional application No. 63/398,484, filed on Aug. 16, 2022.

(51) Int. Cl. A61N 7/00; G02C 11/00

(52) U.S. Cl. CPC A61N 7/00; G02C 11/10; A61N 2007/0026

(57) ABSTRACT

Present implementations can include a high-precision neural stimulation device with a high voltage ASIC with an array of transmit control cells, a 2D array of ultrasound transducers, and a coupling for interfacing the high voltage ASIC to the 2D array, where the high voltage ASIC is disposed to receive and store programmable voltage levels. A contact lens structure can include the high-precision neural stimulation device and one or more fluid channels at least partially surrounding the high-precision neural stimulation device.

### CROSS-REFERENCE TO RELATED PATENT APPLICATIONS

This application claims priority to U.S. Provisional Patent Application Ser. No. 63/398,484, entitled “FLUID CHANNEL STRUCTURE OF NON-INVASIVE PROSTHETIC DEVICE,” filed Aug. 16, 2022, the content of which is hereby incorporated by reference in its entirety and for all purposes as if completely and fully set forth herein.

### FEDERALLY SPONSORED RESEARCH OR DEVELOPMENT

This invention was made with government support under Grant Number R01EY030126, awarded by the National Institutes of Health (NIH). The government has certain rights in the invention.

### TECHNICAL FIELD

The present implementations relate generally to medical devices, and more particularly to a fluid channel structure of a non-invasive neural stimulation prosthetic device.

### BACKGROUND

Interfaces with biological systems and structures, including living biological tissue and in vivo environments, can require invasive medical procedures, implantations, or the like. Such implantation can introduce risk of triggering attendant medical complications or emergencies, and can necessitate further invasive procedures to maintain or replace implanted components.

### SUMMARY

Present implementations can include ultrasonic stimulation devices with high precision and are therefore advantageous for high resolution stimulation applications. An example system can include an array of locally integrated high precision (8-10 bit) Digital to Analog Converters (DACs) efficiently programmed to yield high precision ultrasonic stimulation. The resultant microchip device can be closely integrated with an array of ultrasonic transducers for efficient coupling of electrical signals to respective elements by high density electronic packaging. Devices for neuronal stimulation can focus ultrasound to deliver stimulation energy directly to neurons in a non-invasive manner. Thus, a technological solution for high precision and low power non-invasive neural stimulation prosthetic devices is provided.

Present implementations can include a high-precision neural stimulation device with a high voltage Application Specific Integrated Circuits (ASIC) with an array of transmit control cells, a 2D array of ultrasound transducers, and a coupling for interfacing the high voltage ASIC to the 2D array, where the high voltage ASIC is disposed to receive and store programmable voltage levels.

### BRIEF DESCRIPTION OF THE DRAWINGS

These and other aspects and features of the present implementations will become apparent to those ordinarily skilled in the art upon review of the following description of specific implementations in conjunction with the accompanying figures, wherein:

FIG. 1 illustrates a system in accordance with present implementations.

FIG. 2A illustrates a first view of a first device in accordance with present implementations.

FIG. 2B illustrates a second view of a first device further to the device of FIG. 2A.

FIG. 2C illustrates a third view of a first device further to the device of FIG. 2A.

FIG. 3A illustrates a second device in accordance with present implementations.

FIG. 3B illustrates a second device further to the device of FIG. 3A.

FIG. 4A illustrates a diagram of a property of a device in accordance with present implementations.

FIG. 4B illustrates a diagram of a material property of a device further to the diagram of FIG. 4A.

FIG. 5A illustrates a first timing diagram of a device in accordance with present implementations.

FIG. 5B illustrates a second timing diagram of a device further to the device of FIG. 5A.

FIG. 6 illustrates a device in accordance with present implementations.

FIG. 7 illustrates a device in accordance with present implementations.

FIG. 8 illustrates a device in a particular state in accordance with present implementations.

FIG. 9 illustrates a device in accordance with present implementations.

FIG. 10 illustrates a device in accordance with present implementations.

FIG. 11 illustrates a device in accordance with present implementations.

FIG. 12 illustrates a third timing diagram of a device in accordance with present implementations.

FIG. 13 illustrates a device in accordance with present implementations.

FIG. 14 illustrates a device in accordance with present implementations.

FIG. 15 illustrates a device in accordance with present implementations.

### DETAILED DESCRIPTION

The present implementations will now be described in detail with reference to the drawings, which are provided as illustrative examples of the implementations so as to enable those skilled in the art to practice the implementations and alternatives apparent to those skilled in the art. Notably, the figures and examples below are not meant to limit the scope of the present implementations to a single implementation, but other implementations are possible by way of interchange of some or all of the described or illustrated elements. Moreover, where certain elements of the present implementations can be partially or fully implemented using known components, only those portions of such known components that are necessary for an understanding of the present implementations will be described, and detailed descriptions of other portions of such known components will be omitted so as not to obscure the present implementations. Implementations described as being implemented in software should not be limited thereto, but can include implementations implemented in hardware, or combinations of software and hardware, and vice-versa, as will be apparent to those skilled in the art, unless otherwise specified herein. In the present specification, an implementation showing a singular component should not be considered limiting; rather, the present disclosure is intended to encompass other implementations including a plurality of the same component, and vice-versa, unless explicitly stated otherwise herein. Moreover, applicants do not intend for any term in the specification or claims to be ascribed an uncommon or special meaning unless explicitly set forth as such. Further, the present implementations encompass present and future known equivalents to the known components referred to herein by way of illustration.

Neural stimulation can be accomplished with implanted electrodes through an invasive operation. The implanted electrodes can be prone to degradation of efficacy over time.

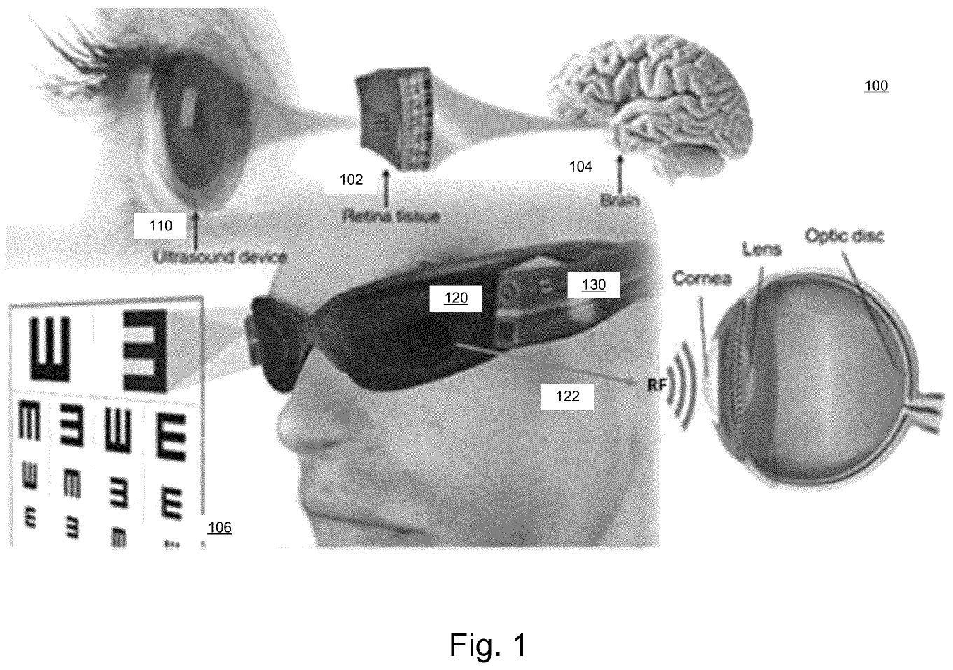

FIG. 1 illustrates a system in accordance with present implementations. In particular, An example visual prosthetic device utilizing a high precision integrated ultrasonic stimulation device is illustrated in FIG. 1. As illustrated by way of example in FIG. 1, an example system 100 can include an ultrasound device 110 operable with a subject (e.g., retina tissue 102 and a brain 104), a wearable optical assembly 120 including the ultrasound device 110, and a wearable camera assembly 130 operable to detect an image 106. For example, the ultrasound device 110 can be attached to or configured to couple with an eye (e.g., a cornea or the retina tissue 102) of the subject.

The wearable camera assembly 130 includes a video camera configured to acquire images or videos (e.g., frames) that are processed locally to determine individual characters that are then sent to a stimulation subsystem of the ultrasound device 110 via RF signals 122 that is mounted to the surface of the eye (e.g., the cornea). An encoding processor translates the visual character information to beamforming matrices of phase delays and voltage levels for the ultrasonic transmitters on the high precision integrated ultrasonic stimulation device. The encoded delay and level matrices can be downloaded to local memory (e.g., a RAM) in the stimulation ASICs for a next stimulation event. A periodic global clock initiates each stimulation event causing all transducer elements in the array of the ultrasound device 110 to be simultaneously stimulated with the correct phase and voltage level to generate the stimulation pattern at the surface of the retina.

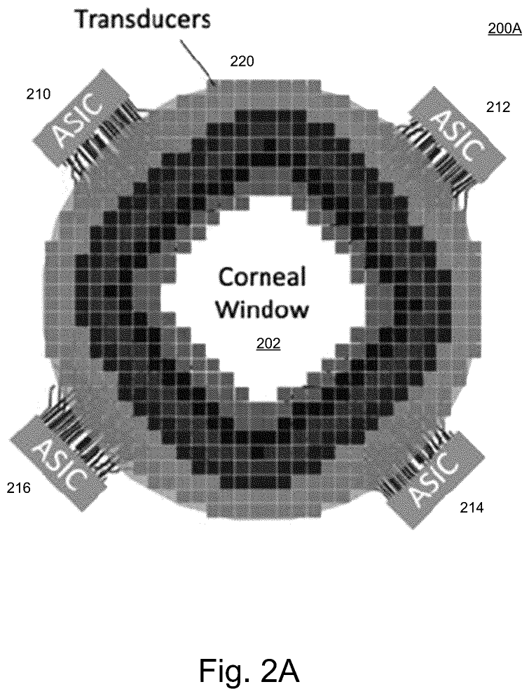

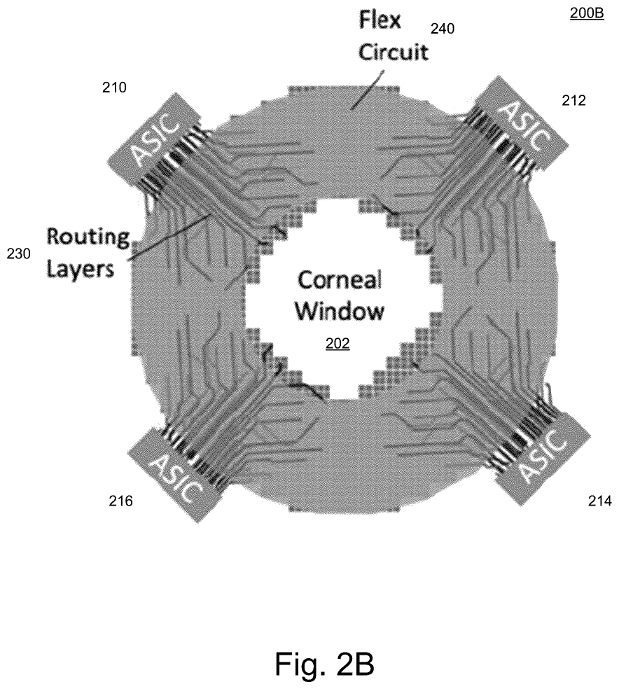

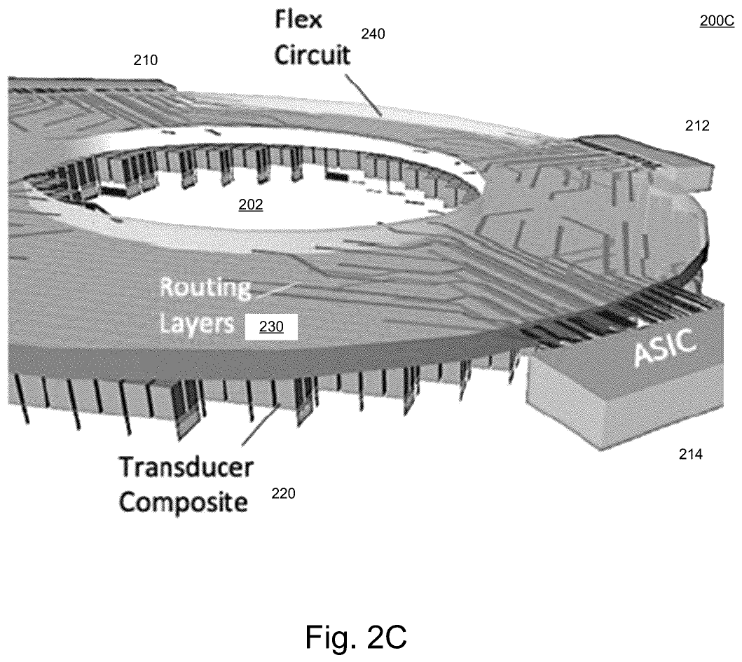

FIGS. 2A-C illustrate example individual stimulation ASICs integrated locally to the stimulation transducer array, via adjacent assembly with an intervening flexible substrate that links signal channels from the ASICs to their respective 2D array ultrasonic elements. Assembly of the ASICs to the flex circuits can be accomplished using electronic assembly techniques including chip on board (COB) wire-bonding and flip-chip reflow bumped assembly. The illustrated stimulation assembly subsystem can be mounted to the surface of an eye of a patient or subject for efficient coupling of ultrasonic energy into the tissue, as shown in FIG. 1. An ultrasonic stimulation device with similar construction to FIG. 1 can be used for stimulation of other types of neurons including those for mounting internal to the skull at the surface of the brain, or subdermally on the outside of the skull. Other stimulation applications may include spinal or limb mounted nerve stimulation for pain management, among others. Direct assembly of the transducer array to the surface of the ASICs and/or utilizing an interposer structure may also be implemented.

FIG. 2A illustrates a first view of a first device in accordance with present implementations. As illustrated by way of example in FIG. 2A, an example device in a top view 200A can include processing devices or processors 210, 212, 214 and 216 and transducers 220 surrounding a corneal window 202. As one example, the processing devices 210, 212, 214 and 216 can include ASICs. A cornea of a subject can be exposed through the corneal window 202 when the device is attached to the cornea of the subject. FIG. 2B illustrates a second view of the first device. As illustrated by way of example in FIG. 2B, an example device in a bottom view 200B can include the processing devices 210, 212, 214 and 216, the transducers 220, and one or more routing layers 230 surrounding the corneal window 202. FIG. 2C illustrates a third view of the first device. As illustrated by way of example in FIG. 2C, an example device in a bottom perspective view 200C can include the processing devices 210, 212 and 214, the transducers 220, the routing layers 230, and one or more traces on one or more of the routing layers 230, surrounding the corneal window 202. As one example, the transducers 220 can include one or more transducer composites. The routing layers 230 can be provided over a flexible circuit 240, which is connected to the transducers 220. The flexible circuit 240 and the routing layers 230 therein connect the transducers 220 to the processing devices 210, 212, 214 and 216.

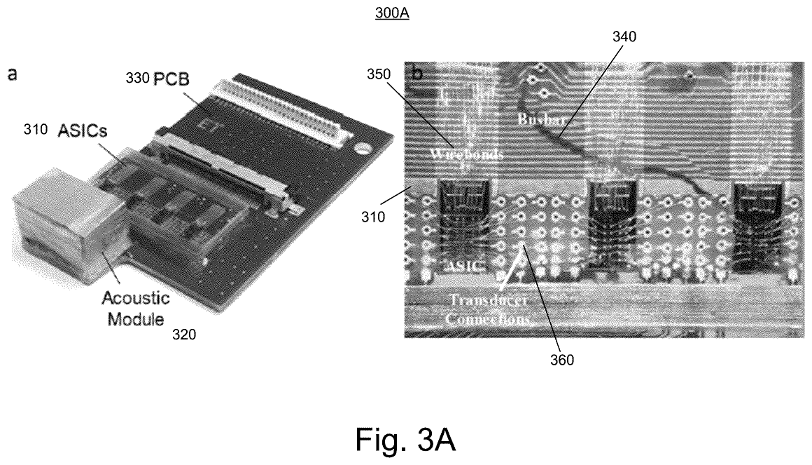

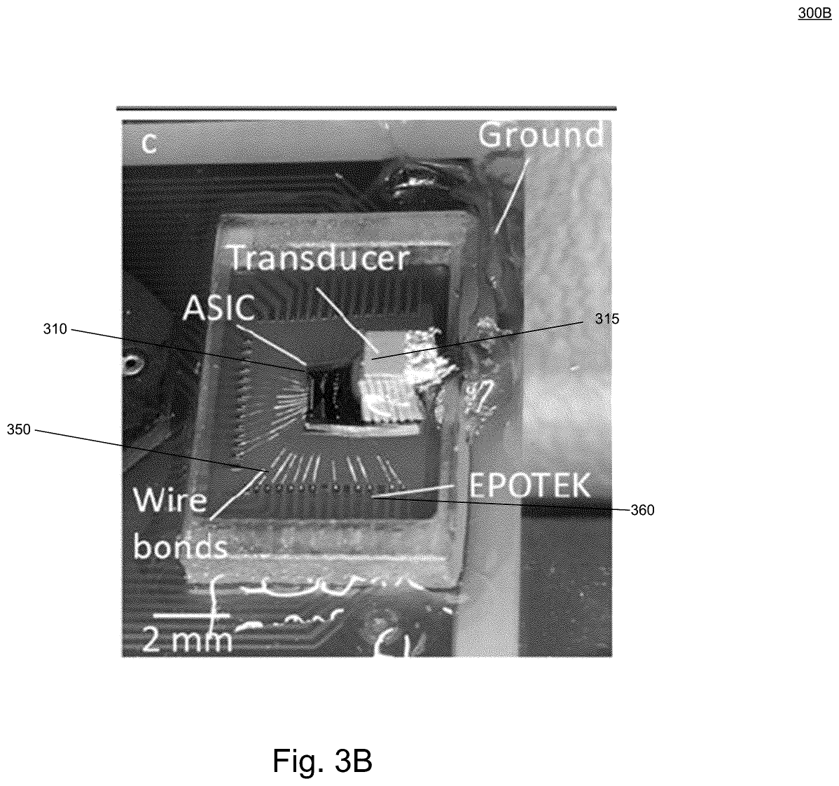

FIGS. 3A-B illustrate two assembly techniques for integrating the ultrasonic stimulation array with ASICs. FIG. 3A illustrates an example “adjacent assembly” used for the illustrated device in FIG. 2 where the ultrasonic array and the ASICs share a common electrical wiring substrate for their communication. FIG. 3B illustrates an example of “direct assembly” where the ultrasonic array is mounted directly on the surface of the ultrasonic stimulation ASICs. Either of these assembly methods can be used depending on the specific constraints of the particular neuronal stimulation application. The transducer arrays themselves may include piezoelectric composite materials and in particular high Kt materials. As one example, PIN-PMN-PT is advantageous in this regard. The transducer arrays may also include capacitive Micromachined Ultrasonic Transducers (cMUTS) or piezo-electric Micromachined Ultrasonic Transducers (pMUT S).

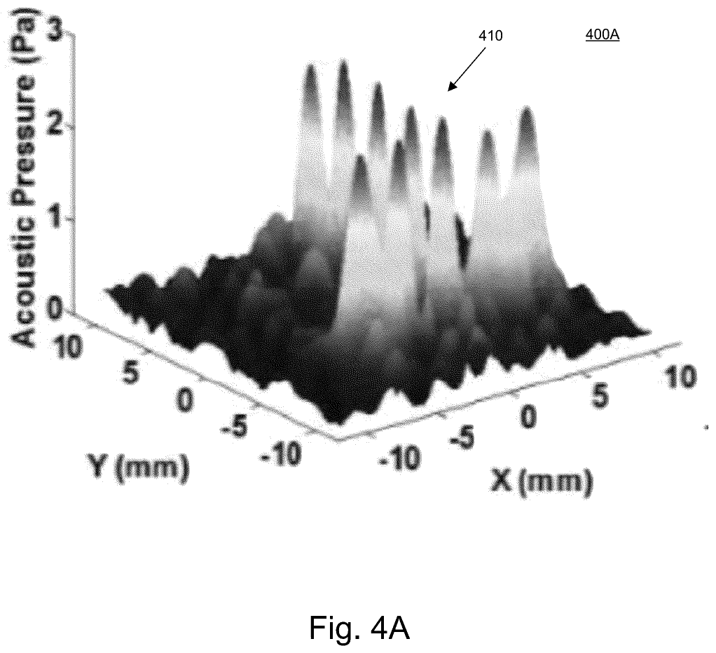

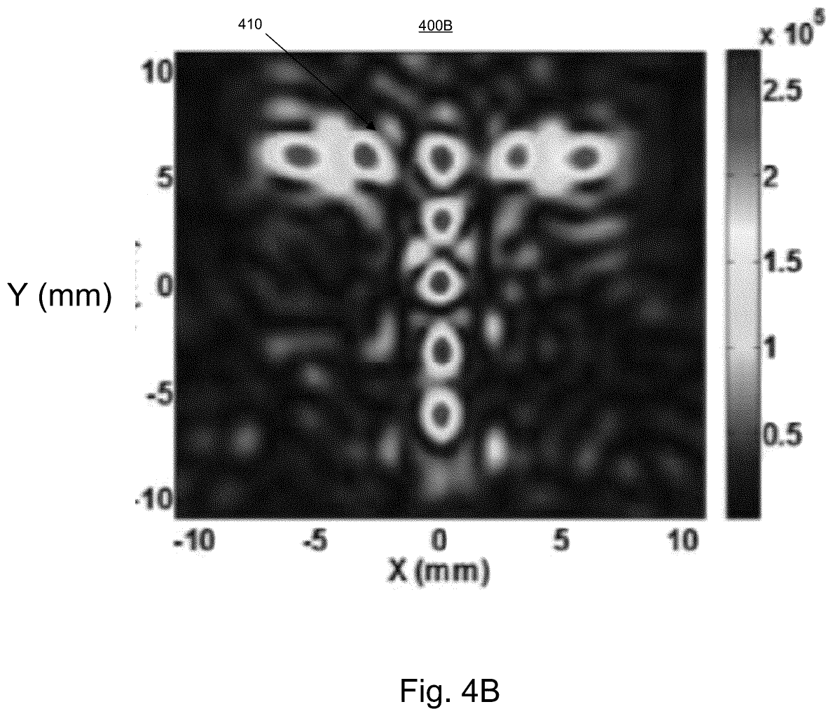

FIG. 3A illustrates a second device in accordance with present implementations. As illustrated by way of example in FIG. 3A, an example device in views 300A (including a perspective view of the second device and a top view of the ASIC 310 of the second device) can include one or more ASICs 310, an acoustic module 320, a printed circuit board (PCB) 330. The ASIC 310 includes a busbar 340, one or more wire bonds 350, and one or more transducer connections 360. FIG. 3B illustrates a second device. As illustrated by way of example in FIG. 3B, an example device in view 300B can include an ASIC 310, a transducer 315, wire bonds 350, a ground and potting epoxy (e.g. EPOTEK) 360. FIGS. 4A-B illustrate the beamforming operation that is used to produce multi-focal patterns at the surface of the neuronal bed for stimulation of complex patterns simultaneously. The technique can include individually defined delays and voltage levels for each transducer element in the ultrasonic transmit matrix.

FIG. 4A illustrates a diagram of an acoustic radiation pattern defined by acoustic pressure in (e.g., in Pa) of a device over an area defined by X and Y (e.g., in mm) in perspective view, in accordance with present implementations. As illustrated by way of example in FIG. 4A, an example diagram 400A can include one or more peaks 410 of the acoustic pressure over the area. FIG. 4B illustrates a diagram of the acoustic radiation pattern defined by acoustic pressure in (e.g., in Pa) of the device over an area defined by X and Y (e.g., in mm) in a top view, in accordance with the present implementations. In peaks 410 shown in the perspective view of FIG. 4A are likewise shown in the top view of FIG. 4B.

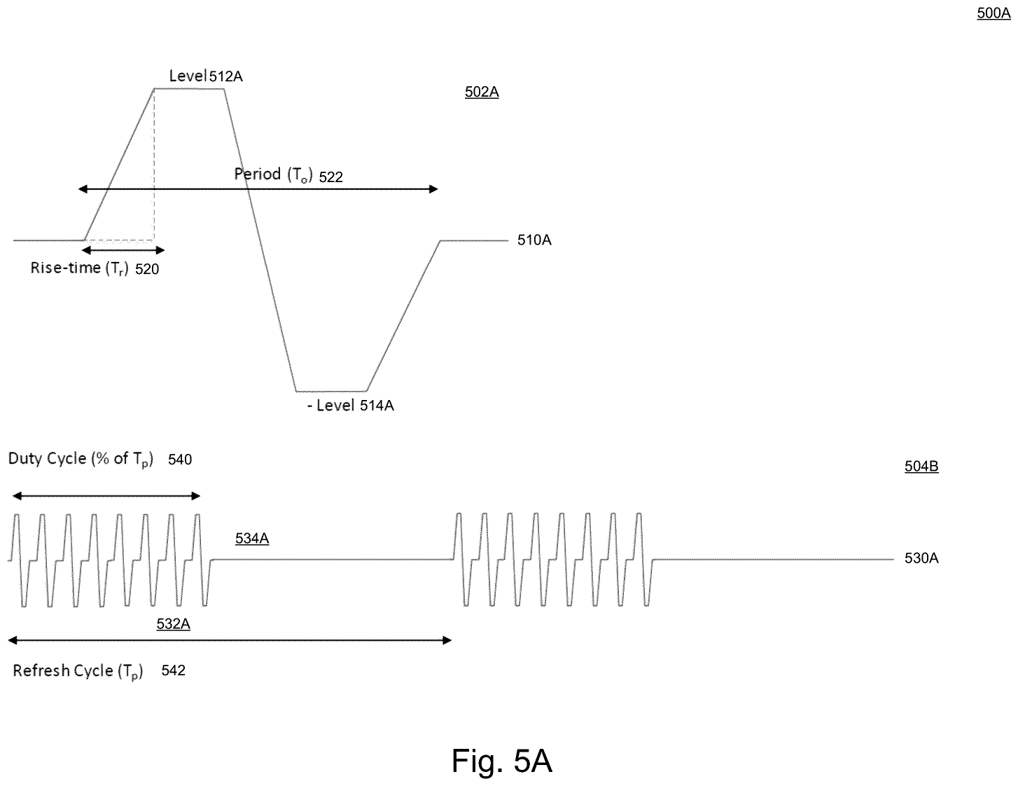

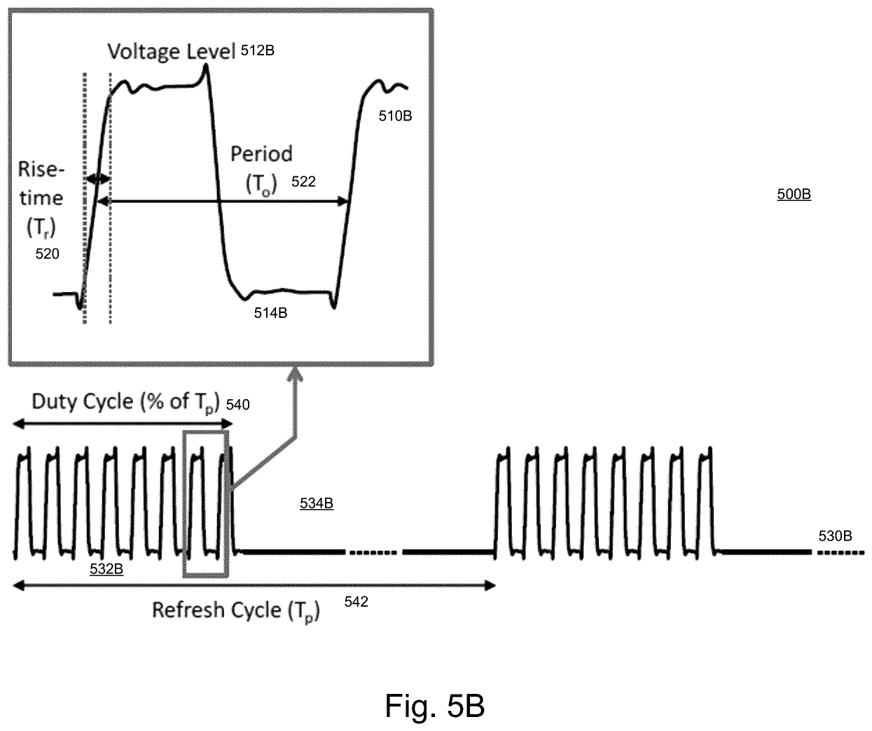

FIGS. 5A-B illustrate example timing in the generated output voltage waveforms for the transmit signals at each of the ultrasonic elements including the period of the transmit clock as well as the refresh rate and duty cycle of the stimulation pulse train patterns.

FIG. 5A illustrates a first timing diagram of a device in accordance with present implementations. As illustrated by way of example in FIG. 5A, an example diagram 500B can include a first waveform 502A and a second waveform 504B. The first waveform 502A can include a first response 510A (e.g., voltage over time) having a first level 512A and a second level 514A. The first level 512A has a rise time 520. The first response 510A has a period 522 including the first level 512A and the second level 514A. The second waveform 504B can include a second response 530A (e.g., voltage over time) having an active portion 532A and an inactive portion 534A. The active portion 532A has a duty cycle 540, A refresh cycle 542 of the second response 530A has a length of an active portion 532A and an inactive portion 534A.

FIG. 5B illustrates a second timing diagram of a device in accordance with present implementations. As illustrated by way of example in FIG. 5B, an example diagram 500B can include a first waveform 510B and a second waveform 530B. The first waveform 510B can include a first response 510B (e.g., voltage over time) having a first level 512B and a second level 514B. The first level 512B has a rise time 520. The first response 510B has a period 522 including the first level 512B and the second level 514B. The second waveform 504B can include a second response 530A (e.g., voltage over time) having an active portion 532B and an inactive portion 534B. The active portion 532B has a duty cycle 540. A refresh cycle 542 of the second response 530B has a length of an active portion 532B and an inactive portion 534B. A portion of the active portion 532B corresponds to the first waveform 510B.

Present implementations can include processing elements or processors, such as ASICs in, for example, AMS 0.35-um high-voltage CMOS process for local control of the ultrasound transducer array. Benefiting from technology scaling, CMOS devices allow massive transmitter (TX) cells integrated monolithically onto a coin-size substrate. Given a target 6 mm×6 mm silicon area, high transmit frequency, i.e., 20 MHz, can be used to reduce the pitch of the array for supporting 256 TX cells each ASIC. As one example (e.g., shown in FIGS. 2A-2C and 3A-3B, four ASICs can be integrated on the top level to have a total of 1024 TX cells.

To realize a highly flexible and precise beam formation, the ASICs can have independent control at each TX cell and hence the associated transducer element. Thus, present implementations can achieve unprecedented control accuracy on the voltage level and the phase delay relative to the global reference clock, with minimum power consumption and area overhead. In addition, ASICs can be powered and controlled using low-channel-count control bus from external control system.

TABLE 1
Example Specifications

| Parameter | Value | |
|---|---|---|
| Active Channels | 1024 | |
| Total ASICs | 4 | |
| Transmit Freq. (Period) | 20 MHz | (50 ns) |
| Refresh Cycle (Rate) | 50 ms | (20 Hz) |
| Voltage Level Resolution | 10 | Bits |
| Delay Time Resolution | 1 | ns |
| Max Delay Time | >100 | ns |
| Duty Cycle | Variable % | |
| Rise Time | 5 | ns |

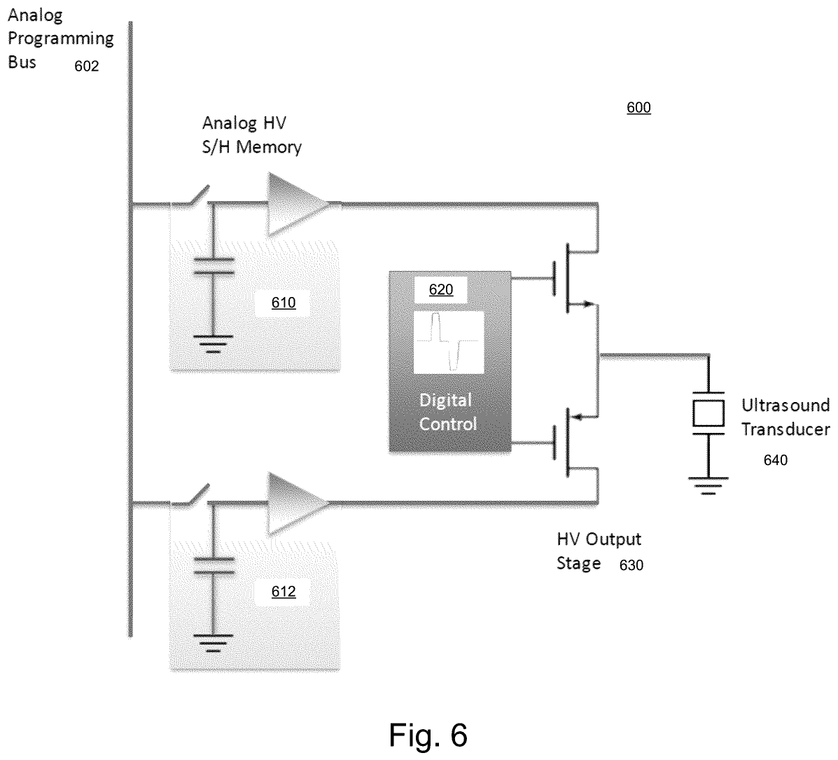

FIG. 6 illustrates a device in accordance with present implementations. As illustrated by way of example in FIG. 6, an example device 600 can include an analog programming bus 602, memory blocks 610 and 612, a digital control block 620, an output stage 630, and an ultrasound transducer 640.

FIG. 6 illustrates the device 600 including a unit cell of the ASIC stimulation array. Locally integrated sample and hold circuits in each cell are disposed to capture a level voltage for the respective element transmit signal for the respective stimulation refresh event. Analog data for each respective ASIC unit cell is programmed serially into the respective sample and hold local analog memory by successively selecting the analog data from a commonly distributed analog programming data bus. The analog data can be distributed in low voltage format (0V to 5V range) for reduced power consumption. In this case, locally integrated buffer amplifiers transform the low voltage level range up to the required high voltage (0V to 50V) for effective stimulation of the transducer elements. Voltage levels can be distributed as high voltage values (0V to 50V range) for simplicity of the local unit cell circuitry implementation. The programming of the entire array of unit cells for each ASIC device (e.g. 256 individual cells), may be accomplished serially from a single analog programming bus, or in parallel using multiple programming busses at each column for more efficient distribution of these signals. In some implementations, voltage levels are encoded digitally and distributed in digital fashion to respective unit cells on a digital bus. A global analog ramping voltage is distributed to all cells along with a digital reference count and the individual unit cells capture and store their respective voltage levels when the global digital count equals their specific stored digital data bit encoded level value.

Based on the acquired and stored analog reference values, the locally integrated digital control circuits in each unit cell can generate a precise peak to peak output voltage at the specific required transmit frequency for correct driving of the 2D array ultrasonic elements. For example, in one application, the transmit frequency is 20 MHz, and therefore the locally integrated digital timing circuitry generates a 20 MHz output pulse that utilizes the locally programmed analog voltage levels to define the height of the output pulse. Both unipolar and bipolar pulse waveform shapes can be used. In the case of bipolar pulse shapes the transmitted voltage waveform may either take the form of symmetric positive and negative voltage levels equal to a single stored analog voltage level, or two locally stored analog voltage levels can be used with the first being transmitted as the positive high level, and the second being transmitted as the negative low level. Return to zero circuitry is also integrated locally. The digital control circuitry can include programmable functionality operable to generate variable pulse trains, where the duty cycle of the pulse train can be independently controlled at every element. Transmit of the pulse is further controlled using locally integrated phase delay circuitry that stores programmed values of delay at each element. Extended pulse trains of multiple independently programmed voltage levels are easily implemented by adding more analog RAM local to each transmitting cell and storing as many reference voltages as required for the respective multiple pulse train values.

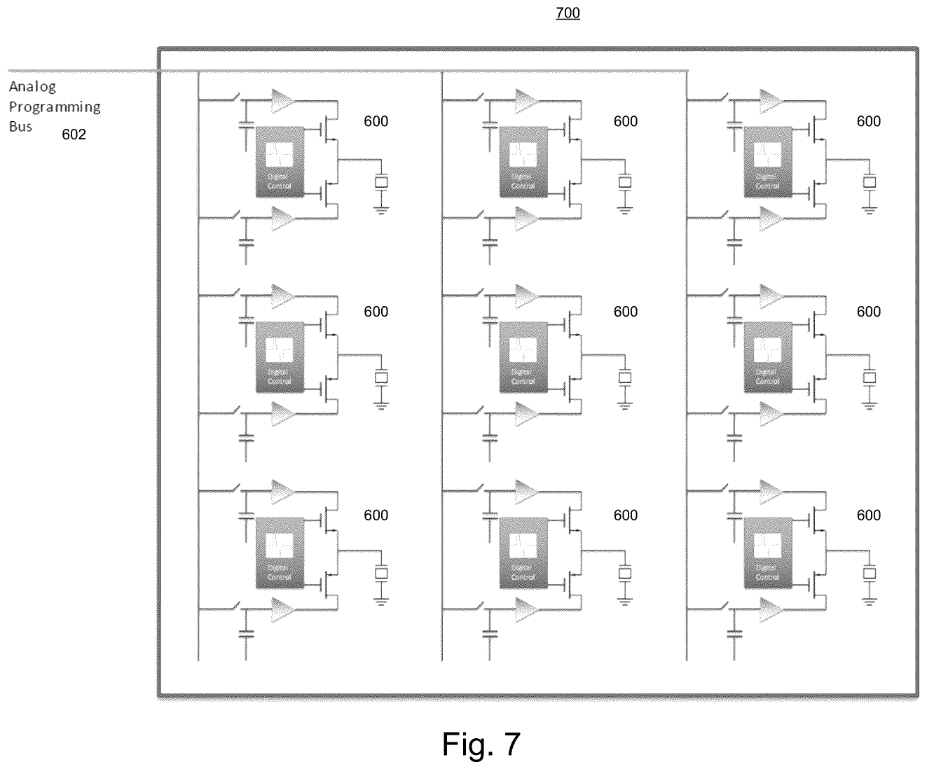

FIG. 7 illustrates a device in accordance with present implementations. As illustrated by way of example in FIG. 7, an example device 700 can include the analog programming bus 602 and one or more of the devices 600 of FIG. 6. The device shown in FIG. 7 includes a large array of the unit cells of FIG. 6 integrated together on a single ASIC to create the stimulation matrix for interface to a 2D ultrasonic array of transducer elements. In some examples, the global DAC is integrated to generate the analog bus values on the same substrate as the transmitting array.

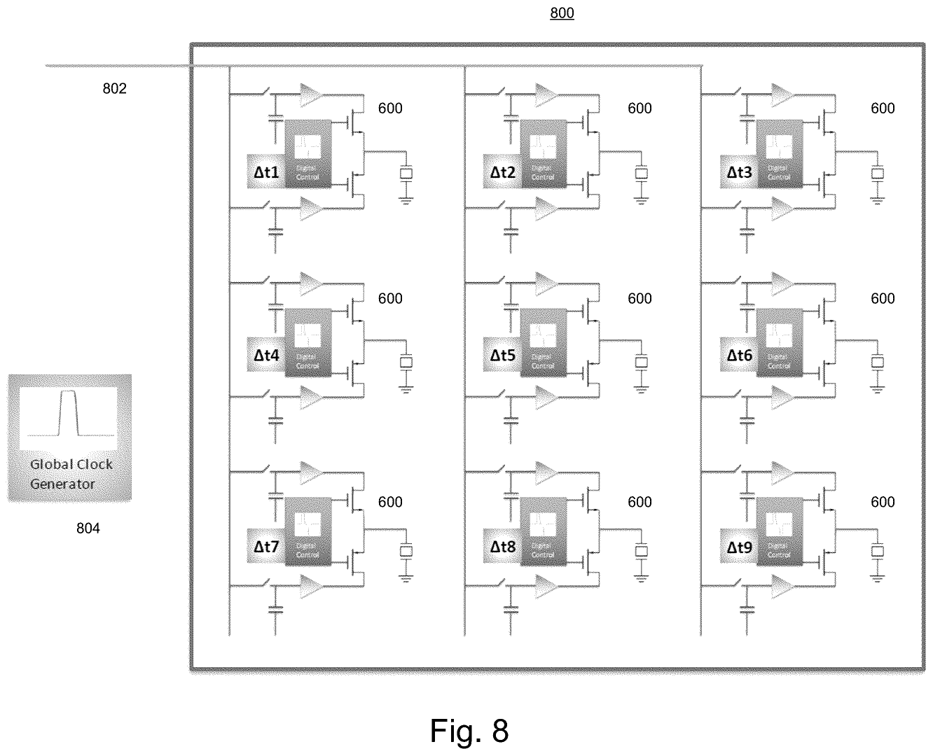

FIG. 8 illustrates a device in a particular state in accordance with present implementations. As illustrated by way of example in FIG. 8, an example device 800 can include an analog programming bus 802, a global clock generator 804, and one or more of the devices 600 of FIGS. 6 and 7. FIG. 8 illustrates a large array of the unit cells of FIG. 7 integrated together on a single ASIC to create the stimulation matrix for interface to a 2D ultrasonic array of transducer elements, and individual respective delays are stored at each unit cell. A time offset Δt is introduced to a digital control block 620 of each of the devices 600.

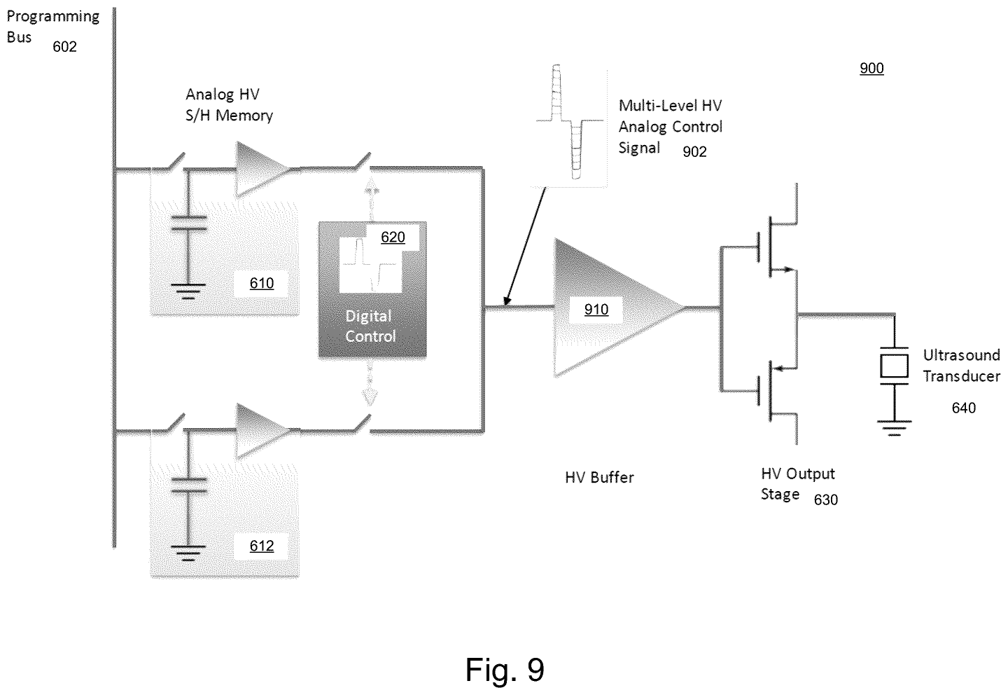

FIG. 9 illustrates a device in accordance with present implementations. As illustrated by way of example in FIG. 9, an example device 900 can include the analog programming bus 602, the memory blocks 610 and 612, the digital control block 620, a buffer 910 operable to receive a multi-level analog control signal 902, the output stage 630, and the ultrasound transducer 640. FIG. 9 illustrates an example similar to the unit cell of FIG. 6. In this case, the combined pulse transmit waveform is generated locally as a low voltage signal and provided to a high voltage buffer amplifier which increases the signal up to the required high voltage stimulation range (0V to 50V) before coupling to an output drive stage and then to the respective ultrasound transducer.

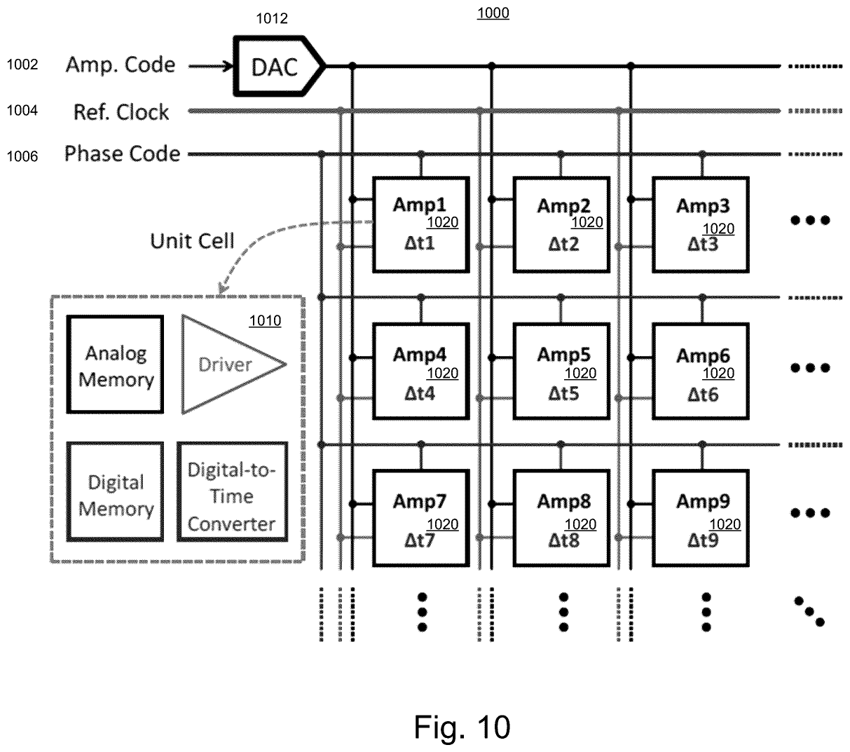

FIG. 10 illustrates an device in accordance with present implementations. As illustrated by way of example in FIG. 10, an example device 1000 can include an amplifier code input 1002, a reference clock input 1004, an a phase code input 1006, a unit cell 1010 including one or more of an analog memory, a driver circuit, a digital memory, and a digital-to-time converter, a digital-to-analog converter 1012, and one or more amplifiers 1020.

ASIC architecture and implementation. Each TX cell can contain a standalone digital-to-analog converter (DAC) 1012 for voltage level control and a digital-to-time converter for phase delay control. The high complexity of such unit TX cell can limit the tuning resolution of voltage level/phase delay (typically 1.5-4 bits) and the size of the beamforming array. Moreover, the signal routing overhead increases linearly with the number of cells and eventually can become the system's bottleneck. The implementations described herein can eliminate a bottleneck with a shared-DAC architecture for the TX array. Leveraging a duty-cycled operation, a global DAC can be shared advantageously among the TX cells and temporarily store different DAC output voltages in the local analog memory of TX cells sequentially when they are not “on-duty.” In this case, only a single wire, i.e., the global DAC output, is sufficient to control the voltage levels for all the TX cells, which advantageously reduces signal routings and enables a large-scale TX array. Similarly, the phase code bus is shared by all the TX cells. A local digital memory can fetch and store the phase code for each TX cell at a given time. The global reference clock can be properly buffered and distributed to each TX cell for minimum timing skews. Present implementations can integrate a high-precision DAC into the ASIC, such as using a silicon-proved hybrid DAC structure. The delta-sigma modulator in the hybrid DAC can achieve >12-bit accuracy with reduced analog complexity. The extra two bits can calibrate the distortion and mismatch errors between TX cells.

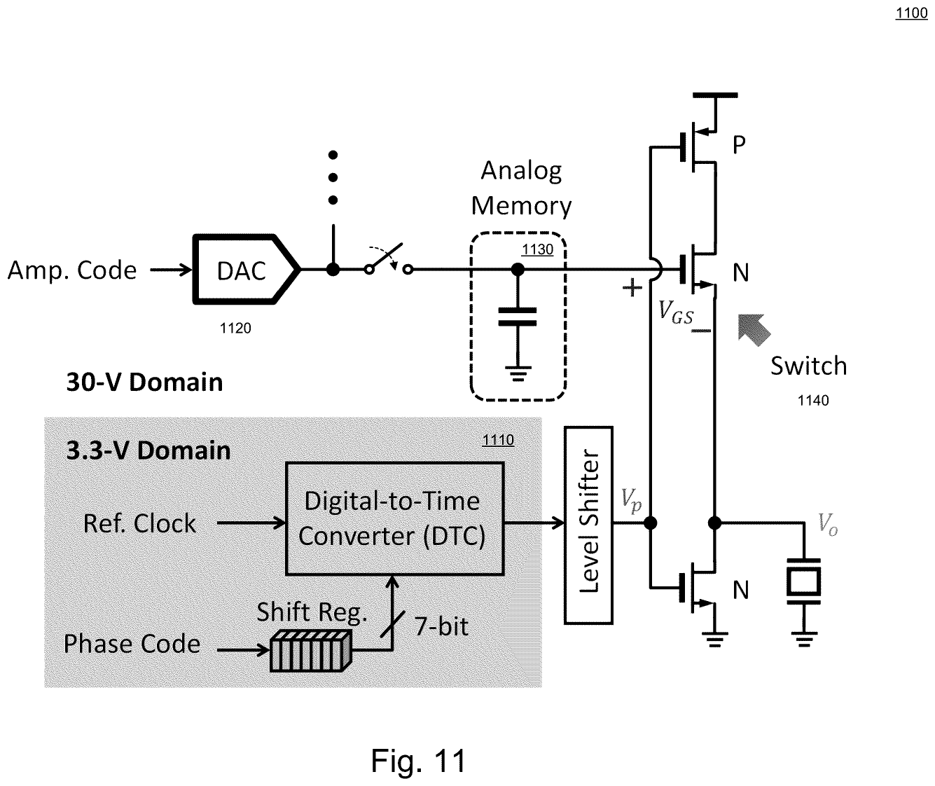

FIG. 11 illustrates a device in accordance with present implementations. As illustrated by way of example in FIG. 11, an example device 1100 can include a digital-to-time converter circuit 1110, a digital-to-analog converter 1120, an analog memory 1130, and a switch circuit 1140.

The device shown in FIG. 11 is an example implementation of the unit TX cell. A sample-and-hold is used as the analog memory for holding the voltage level, including a bootstrapped switch and a capacitor. Compared to the structure with digital memory followed by a DAC, the capacitor-based analog memory significantly reduces the design complexity as well as the implementation cost for the case with such high-resolution (>10 bits) voltage levels. A modified CMOS inverter with an inserted N-type switch controlled by the store voltage level can drive the transducer.

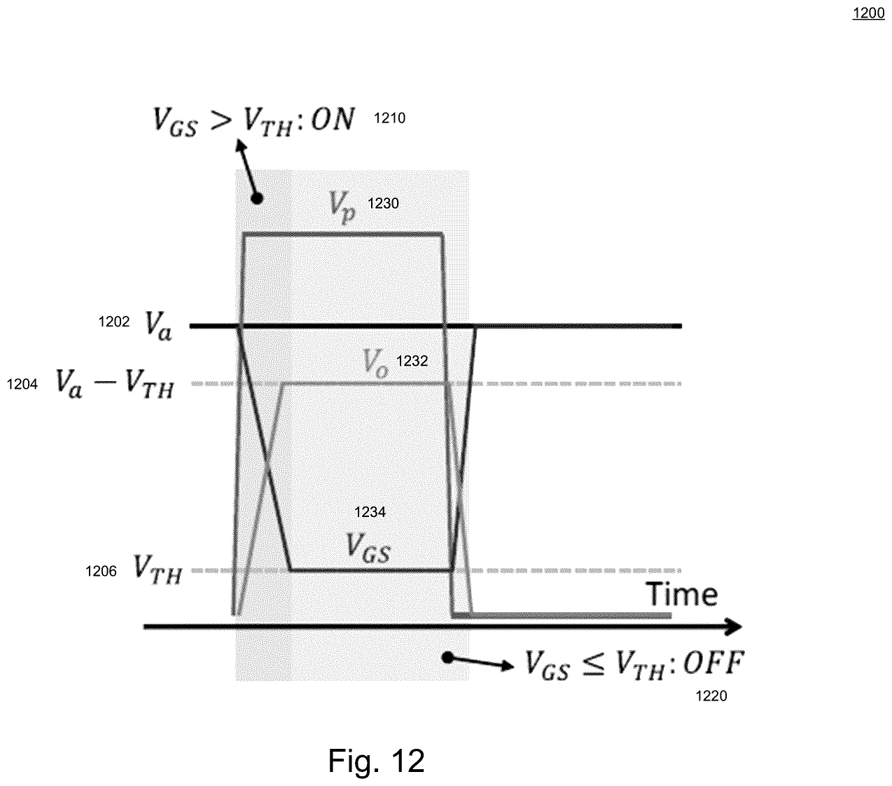

FIG. 12 illustrates a third timing diagram of a device in accordance with present implementations. As illustrated by way of example in FIG. 12, an example timing diagram 1200 can include a first period 1210, a second period 1220, a first input voltage 1202, a second input voltage 1204, a third input voltage 1206, a first voltage curve 1230, a second voltage curve 1232, and a third voltage curve 1234.

When Vp turns on the PMOS transistor, V0 starts to be charged up by the 30-V supply and automatically turns the N-type switch off when VGS reaches the threshold voltage Vth, i.e., V0=Va−Vth. Instead of active buffers, such as amplifier and source follower, the TX cell mainly includes passives and switches, facilitating high-density arrays and associated design and layout automation. The switches and the capacitor can be properly sized to ensure ignorable leakage from the capacitor. Note that, a design example for unipolar pulse generation is shown since the artifacts of such pulses are tolerable in our TX application. That said, the complimentary structure of the TX cell makes it readily extendible for bipolar pulse generation with negligible implementation overhead. The phase delay tuning circuits operate at 3.3-V supply for power saving and better timing control. The following voltage level shifter converts the 3.3-V pulses to 30-V rail-to-rail for effectively driving the transducer.

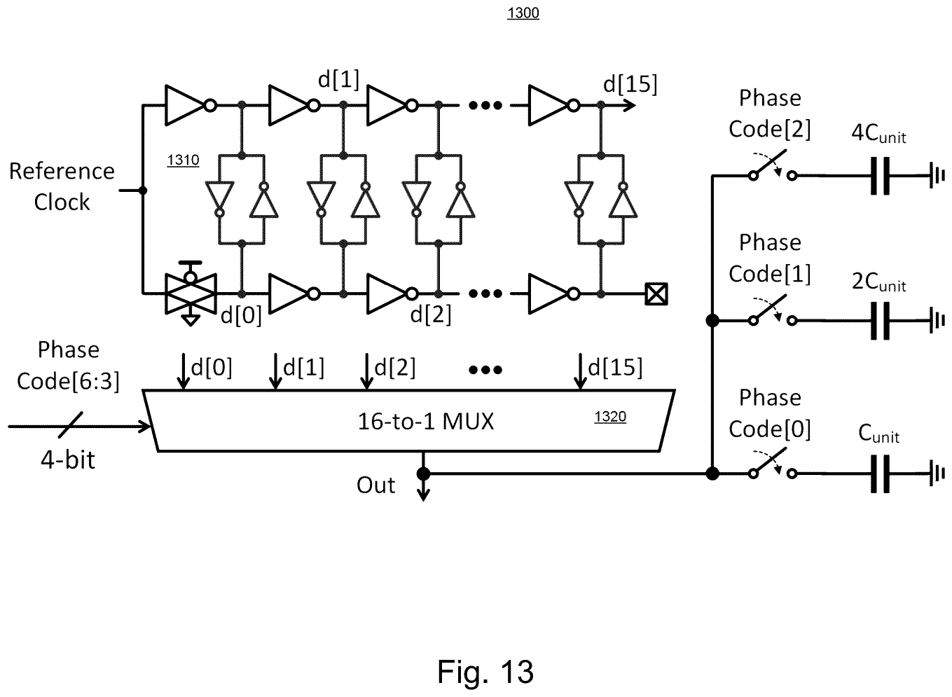

FIG. 13 illustrates a device in accordance with present implementations, including an example two-stage segmented Digital to Time Converter (DTC). The segmentation can optimize tradeoff between area/power and nonlinearity. The first stage can include an inverter-based delay line for creating different delay levels and a multiplexer (MUX) for selecting the delayed signal based on the MSBs of the phase code. Present implementations can reduce the delay tuning resolution to one inverter delay. Extra cost introduced by the coupling inverters is negligible, due to their small size. A group of 2-to-1 MUXs connected in a tree structure can be used for the delayed signal selection. The second stage is a 3-bit binary-weighted DTC implemented with switched capacitors. The binary weighting can be realized by sizing the capacitors. Compared with the delay line, the switched capacitor DTC can achieve fine delay tuning; hence we use it as the LSB control. In this project, the DTC is tuned by a 7-bit phase code, i.e., Phase Code[6:0], and supports a delay tuning resolution of around 1 ns under a 3.3-V supply voltage.

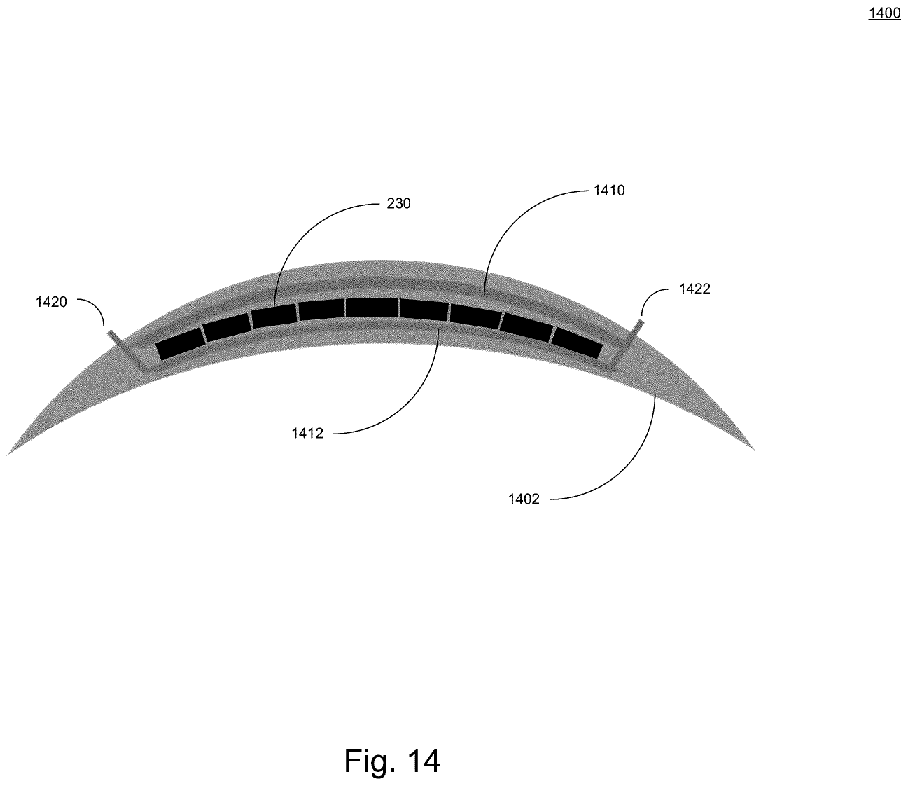

FIG. 14 illustrates a device in accordance with present implementations. As illustrated by way of example in FIG. 14, an example device 1400 in cross-sectional view can include the routing layers 230, an enclosure 1402, an upper fluid chamber 1410, a lower fluid chamber 1412, a first fluid channel 1420 and a second fluid channel 1422. The enclosure 1402 can include a solid material and can correspond to a lenticular shape. For example, the enclosure 1402 can have one or more of a shape and a size corresponding to a contact lens compatible with a human eye. The enclosure 1402 can include a flexible material and can be formed to at least partially isolate the routing layers 230 from an ambient environment or an in vivo environment.

The upper fluid chamber 1410 can include an opening formed at least partially within the enclosure 1402. For example, the upper fluid chamber 1410 can include one or more of a tube, track, rut, pipe, or any combination thereof. The upper fluid chamber 1410 can surround a cavity in the enclosure 1402. The routing layers 230 can be disposed between the upper fluid chamber 1410 and a surface of the enclosure 1402 contactable with a biological surface. The lower fluid chamber 1412 can include an opening formed at least partially within the enclosure 1402. For example, the lower fluid chamber 1412 can include one or more of a tube, track, rut, pipe, or any combination thereof. The lower fluid chamber 1412 can surround a cavity in the enclosure 1402. The upper fluid chamber 1410 can be disposed between the routing layers 230 and a surface of the enclosure 1402 contactable with a biological surface.

The first fluid channel 1420 and the second fluid channel 1422 can include an opening formed between one or more of the upper fluid chamber 1410, the lower fluid chamber 1412, an ambient environment, and an in vivo environment. For example, the first fluid channel 1420 and the second fluid channel 1422 can provide an inlet or outlet for fluid generated by or disposed on the enclosure 1402. Fluid, for example, can include natural tear fluid, artificial tea fluid, or any combination thereof. Fluid entering the upper fluid chamber 1410 or the lower fluid chamber 1412 can be circulated around the routing layers 230 to absorb waste heat from the routing layers 230.

Thus, this technical solution can include a technical improvement of providing cooling of electronic components in an in vivo environment by fluids present in the in vivo environment. The technical solution can include an ultrasound array that can be attached to the cornea for long-time wearing, like a contact lens, to stimulate and image the eyeball, especially the retina. The technical solution can be combined with light sources or sonogenetics. Ultrasound can stimulate retina as a non-invasive retina prosthesis, with or without sonogenetics. Various ultrasound transducers and arrays cannot provide for retina stimulation, based on characteristics including size, shape, frequency, or power, that are not compatible with in vivo environments. A technical solution including an ultrasound array can be used with or without sonogenetics. The array can also combine with light sources to do light/ultrasound multimodal imaging and stimulation.

An example array has an outer diameter from 12-24 mm and a curvature with the radius of 20-25 mm. The array can have a central window with a diameter of 8-15 mm to avoid affecting eye lens, and/or work with light sources. The center frequency of array can range, for example, from 3 MHz to 60 MHz. The ultrasound array can be covered in contact lens materials, including polymer, silicone, and/or hydrogel. The cover can be manufactured with curvature too to match the curvature of an eyeball or portion thereof. Water cooling channels can be integrated in the contact lens cover to remove the heating on the surface of the array. Piezoelectric material of the array can be PZT, PMN-PT, PIN-PMN-PT, LNO and any other piezoelectric material.

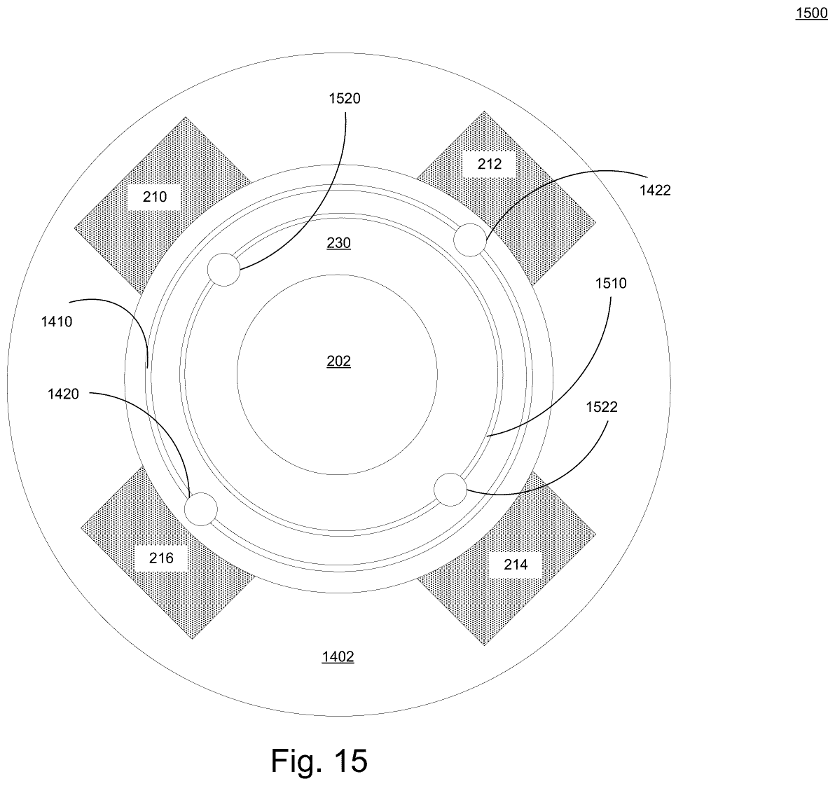

FIG. 15 illustrates a twelfth device in accordance with present implementations. As illustrated by way of example in FIG. 15, an example device in a plan view 1500 can include the processing devices 210, 212, 214 and 216, the routing layers 230 surrounding the corneal window 202, the enclosure 1402, the upper fluid chamber 1410, the first fluid channel 1420, the second fluid channel 1422, a second upper fluid chamber 1510, a third fluid channel 1520, and a fourth fluid channel 1522.

The upper fluid chamber 1410 and the second upper fluid chamber 1510 can be arranged within the enclosure 1402 in concentric rings, circles, or the like, for example. The upper fluid chamber 1410 and the second upper fluid chamber 1510 can each surround cavities contactable with an ambient environment or an in vivo environment by one or more of the first fluid channel 1420, the second fluid channel 1422, the third fluid channel 1520, and the fourth fluid channel 1522. The second upper fluid chamber 1510 can be disposed over a second lower fluid chamber.

A processing device, processing circuit, processor, or processing unit described herein can execute one or more instructions. The processing device can obtain one or more of the instructions via at least one of the system memory. The processing device can include an electronic processor, an integrated circuit, or any combination thereof, for example, including one or more of digital logic, analog logic, digital sensors, analog sensors, communication buses, volatile memory, nonvolatile memory, or any combination thereof. The processing device can include but is not limited to, at least one ASIC, microcontroller unit (MCU), microprocessor unit (MPU), central processing unit (CPU), graphics processing unit (GPU), physics processing unit (PPU), embedded controller (EC), programmable gate array (PGA), field-programmable gate array (FPGA), or any combination thereof, for example. The processing device can include a memory operable to store or storing one or more instructions for operating components of the processing device and operating components operably coupled to the processing device. The one or more instructions can include at least one of firmware, software, hardware, operating systems, embedded operating systems, or any combination thereof.

The system memory can store data associated with any example processing system described herein. The system memory can include a hardware memory device to store binary data, digital data, or any combination thereof. The system memory can include one or more electrical components, electronic components, programmable electronic components, reprogrammable electronic components, integrated circuits, semiconductor devices, flip flops, arithmetic units, or any combination thereof. The system memory can include at least one of a non-volatile memory device, a solid-state memory device, a flash memory device, and a NAND memory device. The system memory can include one or more addressable memory regions disposed on one or more physical memory arrays. For example, a physical memory array can include a NAND gate array disposed on a particular semiconductor device, integrated circuit device, printed circuit board device, or any combination thereof.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative implementations has been presented for purposes of illustration and of description. It is not intended to be exhaustive or limiting with respect to the precise form disclosed, and modifications and variations are possible in light of the above teachings or may be acquired from practice of the disclosed implementations. It is intended that the scope of the invention be defined by the claims appended hereto and their equivalents.

What is claimed is:

1. A high-precision neural stimulation system comprising
a high voltage Application Specific Integrated Circuit (ASIC) with an array of transmit control cells; and
a 2D array of ultrasound transducers coupled to the high voltage ASIC,
wherein the high voltage ASIC is disposed to receive and store programmable voltage levels.

2. The system of claim 1, further comprising a local storage of individual phase delays for each respective transducer channel.

3. The system of claim 1, wherein at least one of:
the 2D array of ultrasound transducers is coupled to the high voltage ASIC via an electrical routing substrate;
the 2D array of ultrasound transducers is coupled to the high voltage ASIC via a vertically integrated assembly.

3. The system of claim 1, wherein at least one of:
the ultrasound transducers are micromachined; or
the ultrasound transducers are sectioned using a dicing saw.

4. The system of claim 1, wherein each transmit control cell of the array of transmit control cells comprises a locally integrated analog memory capturing a globally distributed programmable level voltage.

5. The system of claim 4, wherein at least one of:
the high voltage ASIC comprises an analog memory having capacitive sample and hold circuitry;
the analog memory is programmed by sequential sampling of analog voltage levels on a globally distributed analog programming bus;
the analog memory is programmed by sequential sampling of analog voltage levels on multiple globally distributed analog programming busses; or
the analog memory is programmed by sampling of a globally distributed ramping analog voltage based on a digitally programmed time step value at each unit cell.

6. The system of claim 1, wherein at least one of:
the programmable voltage levels are generated by a locally integrated noise-shaping DAC at each element; or
the programmable voltage levels are generated by a single locally integrated noise-shaping DAC for the entire array.

7. The system of claim 1, wherein each transmit control cell comprises multiple locally integrated analog memory cells capturing multiple globally distributed programmable level voltages.

8. The system of claim 7, wherein extended pulse trains of multiple independently programmed voltage levels are implemented by transmitting in turn pulses that are scaled to match the respective individual voltage levels stored in the multiple individual local analog memory cells.

9. A high-precision neural stimulation system comprising:
a high voltage ASIC with an array of transmit control cells; and
a 2D array of ultrasound transducers coupled to the high voltage ASIC,
wherein the transmit control cells comprise locally integrated pulse width modulation circuits.

10. The system of claim 9, wherein the pulse width modulation circuits are calibrated to create a desired output voltage level based on the measured respective impedance of the transducer element at the given channel.

11. The system of claim 9, wherein a globally distributed analog test bus is used to sample the output of each transmitter.

12. The system of claim 9, wherein the sampled output values are used to pre-warp the programmable level values to calibrate the output of the respective transmitters.

13. A high-precision neural stimulation system comprising:
a high voltage ASIC with an array of transmit control cells; and
a 2D array of ultrasound transducers coupled to the high voltage ASIC,
wherein the transmit control cells comprise locally integrated noise-shaping DACs.

14. The system of claim 13, wherein a sampled programmable level voltage is in at least one of:
a range of 0V to 5V; or
a range of 0V to 50V.

15. The system of claim 13, further comprising a high voltage amplifier configured to generate an output drive signal in a range of 0V to 50V in response to a sampled programmable level voltage.

16. The system of claim 13, wherein
a high voltage buffer is used to isolate a sampled programmable level voltage from the output of the cell; or
the system further comprising high voltage switches that are configured to be sequentially actuated to couple the buffered sampled programmable level voltages to the output.

17. The system of claim 17, further comprising a digitally programmable pulses train sequencing cell.

18. A contact lens structure, comprising:
the high-precision neural stimulation system of claim 1; and
one or more fluid channels at least partially surrounding the high-precision neural stimulation device.

19. The contact lens structure claim 18, further comprising:
one or more fluid inlets defining an opening between one or more of the fluid channels and an environment.

20. The contact lens structure claim 19, the environment comprising one or more of an ambient environment and an in vivo environment.
